(19) United States
(12) Patent Application Publication
(10) Pub. No.: US 2023/0201285 A1
(43) Pub. Date: Jun. 29, 2023

(54) GRANULAR COMPOSITION PROVIDING WATER DISPERSIBLE CANNABINOIDS AND METHODS OF MAKING THE SAME

(71) Applicant: Pegasus Laboratories, Inc., Shawnee, KS (US)

(72) Inventors: Angela Rea-Ramsey, Gulf Breeze, FL (US); Scott Howard, Pace, FL (US)

(21) Appl. No.: 18/067,150

(22) Filed: Dec. 16, 2022

Related U.S. Application Data
(60) Provisional application No. 63/294,595, filed on Dec. 29, 2021.

(51) Int. Cl. A61K 36/185; A61K 31/05; A61K 9/16; A61K 9/20; A61K 9/00; A23K 50/40; A23K 40/10

(52) U.S. Cl. CPC A61K 36/185; A61K 31/05; A61K 9/16; A61K 9/20; A61K 9/0056; A23K 50/40; A23K 40/10

(57) ABSTRACT

The present invention relates to compositions providing water-soluble or water-dispersible cannabinoids and to methods of producing and administering the same. These compositions improve the versatility and absorption of water insoluble cannabinoid extracts, derivatives, and isolates. When added to water, these compositions are capable of forming a stable emulsion and can provide a concentration of cannabinoids of up to 4 mg/mL without the use of significant heat, ultrasonication, spray drying, or high-pressure extrusion.

### CROSS-REFERENCE TO RELATED APPLICATIONS

The present application claims the priority benefit of U.S. Provisional Patent Application Ser. No. 63/294,595, filed Dec. 29, 2021, entitled GRANULAR COMPOSITION PROVIDING WATER DISPERSIBLE CANNABINOIDS AND METHODS OF MAKING THE SAME, incorporated by reference in its entirety herein.

### BACKGROUND OF THE INVENTION

### Field of the Invention

The present invention relates to compositions providing water-soluble or water-dispersible cannabinoids and to methods of producing and administering the same. When added to liquids, especially water, these compositions are capable of forming a stable emulsion and can provide a high concentration of cannabinoids without the use of significant heat, ultrasonication, spray drying, or high-pressure extrusion.

### Description of Related Art

Cannabinoid derivatives isolated from the plants of the genus Cannabis sp., e.g., cannabidiol (CBD), are commonly used holistically to treat many common ailments, such as pain or anxiety. However, cannabinoid extracts and isolates are not water-soluble as processed and naturally exist as substantially non-polar molecules. As a result, these extracts and isolates are typically prepared in, and orally administered in an oil base or tincture. Some commonly used oils as oral delivery systems include vegetable oils, coconut oil, and medium chain triglycerides (MCT oil). These oil solutions are slower to be absorbed than a water-based preparation.

Furthermore, current commercially available water-based preparations have limited solubility, and, if they are somewhat soluble, the water-based preparations require significant heat (i.e., over ˜60° C.), ultrasonication, spray drying, or high-pressure extrusion to create an emulsion. Often, in these water-based preparations, the cannabinoids may not evenly disperse into water, forming an unstable oil emulsion, forming insoluble cannabinoid particles that visibly settle to the bottom of the water, or separating as an oily film on the top of the water. In addition, the commercially available water-based preparations are also very dilute. In fact, final preparations result in less than 0.1 mg/mL of CBD in an aqueous base.

Therefore, there is a need for a composition that improves the versatility and bioavailability of water insoluble cannabinoid extracts, derivatives, and isolates. There is also a need for a composition that provides substantially higher solubility of cannabinoids in liquids, such as water, without the use of additives, prolonged processing, or significant energy input, such as heat or high shear mixing.

### SUMMARY OF THE INVENTION

The present invention is broadly concerned with a composition comprising, consisting essentially of, or consisting of about 5% to about 20% by weight of one or more cannabinoids, about 5% to about 20% by weight of at least one emulsifier; about 0.5% to about 5% by weight of at least one solvent, and about 30% to about 85% by weight of at least one water-soluble bulking agent, wherein each % by weight is based upon the total weight of said composition taken as 100% by weight. In preferred embodiments, the one or more cannabinoids is selected from the group consisting of cannabidiols, cannabigerols, cannabichromenes, cannabinols, and delta 9-tetrahydrocannabinols. In other preferred embodiments, the composition is capable of forming a stable emulsion with water at a weight ratio of from about 1:25 to about 1:35. In more preferred embodiments, at least about 50% of the one or more cannabinoids within the composition is bioavailable within 1 hour after ingestion. In most preferred embodiments, when fed an effective amount of the composition to a canine, about 100 to about 140 ng/mL of the one or more cannabinoids are present in the canine's blood within 1 hour.

The present invention is also directed toward methods of producing the compositions. In one or more embodiments, the method comprises (a) combining at least one primary solvent, at least one emulsifier, at least one co-solvent, and one or more cannabinoids together to form a liquid phase; (b) mixing the liquid phase with a dry phase to form a cannabinoid composition, the dry phase comprising at least one water-soluble bulking agent; and (c) drying the composition. In preferred embodiments, the method further comprises (d) after step (c), oscillating the composition through a mesh screen to form a granular composition. In other preferred embodiments, the cannabinoid composition formed in step (b) comprises from about 30% to about 50% by weight of the liquid phase and from about 50% to about 70% by weight of the dry phase, wherein each % by weight is based upon the total weight of the cannabinoid composition formed in step (b).

In another aspect, the present invention is directed toward a liquid suspension. The liquid suspension comprises about 1 part by weight of the composition dispersed in no more than about 50 parts by weight of a liquid carrier, wherein the suspension has a concentration of about 0.1 mg/mL to about 5 mg/mL of cannabinoids.

Finally, the present invention is directed toward methods of administering cannabinoids to an animal. The methods comprise feeding the animal an effective amount of the composition or the liquid suspension.

### BRIEF DESCRIPTION OF THE DRAWINGS

FIG. 1 is a process diagram illustrating a method of producing the compositions according to one embodiment of the present invention.

FIG. 2 is a chart illustrating the average cannabidiol plasma concentration (ng/mL) in 18 beagle dogs, measured over the course of 24 hours during an in-vivo pharmacokinetic crossover study.

### DETAILED DESCRIPTION

The present invention concerns novel compositions and methods of making and administering the compositions. Also described are novel liquid suspensions comprising the compositions that have a concentration of about 0.1 mg/mL to about 5 mg/mL of cannabinoids and methods of administering these suspensions.

### Water-Dispersible Cannabinoid Compositions

In one or more embodiments, the compositions comprise one or more cannabinoids, at least one emulsifier, and at least one co-solvent, which are each dissolved or dispersed in a primary solvent.

### 1. Ingredients

In most embodiments, the compositions comprise one or more cannabinoids, which may be in a dry or liquid form, preferably a dry form. The one or more cannabinoids may be exogenous cannabinoids (exocannabinoids), such as phytocannabinoids (e.g., those derived from the cannabis plant), or endogenous cannabinoids (endocannabinoids), which are substances produced from within a body of an animal that activate cannabinoid receptors within the body. The exocannabinoids and endocannabinoids can be naturally derived, such as through extraction and isolation from plant and animal sources or they may be synthetically produced. Examples of endocannabinoids include cannabimimetic fatty acid derivatives, such as, but not limited to, arachidonic acid derivatives including arachidonoylethanolamide (anandamide), 2-arachidonoyl glycerol (2-AG), palmitoylethanolamide, or methanandamide.

In preferred embodiments, the one or more cannabinoids are natural exogenous cannabinoids extracted and/or isolated from plants (e.g., phytocannabinoids), preferably the Cannabis plant, or synthetic exogenous cannabinoids. In these such embodiments, the one or more cannabinoids may be cannabinoid acids, such as, but not limited to, cannabidiolic acid (CBDA), cannabigerolic acid (CBGA), cannabichromenenic acid (CBCA), cannabinolic acid (CBNA), cannabidivarinic acid (CBDVA), cannabichromevarinic acid (CBCVA), cannabigerovarinic acid (CBGVA), delta 8-tetrahydrocannabinolic acid (D8THCA), delta 9-tetrahydrocannabinolic acid (D9THCA), delta 10-tetrahydrocannabinolic acid (D10THCA), and tetrahydrocannabivarinic acid (THCVA). In these such embodiments, the one or more cannabinoids may also be other cannabinoid compounds, such as, but not limited to, cannabidiol (CBD), cannabigerol (CBG), cannabichromene (CBC), cannabinol (CBN), cannabidivarin (CBDV), cannabichromevarin (CBCV), cannabigerovarin (CBGV), delta 8-tetrahydrocannabinol (D8THC), delta 9-tetrahydrocannabinol (D9THC), delta 10-tetrahydrocannabinol (D10THC), and tetrahydrocannabivarin (THCV).

In most preferred embodiments, the one or more cannabinoids are selected from the group consisting of CBD, CBG, CBC, CBN, D8THC, D9THC, and D10THC. In most preferred embodiments, the one or more cannabinoid is CBD or D9THC. In embodiments where the one or more cannabinoid is CBD, the CBD may be in the form of CBD isolate, broad-spectrum CBD, or full-spectrum CBD. As used herein, the term “CBD isolate” means a form of CBD being at least 98% pure CBD and containing no other Cannabis plant compounds. As used herein, the term “broad-spectrum CBD” means a form of CBD containing one or more other Cannabis plant compounds except for THC. As used herein, the term “full-spectrum CBD” means a form of CBD containing one or more other Cannabis plant compounds, including up to 0.3% of D9THC. In embodiments where the one or more cannabinoid is D9THC, the D9THC may be in the form of D9THC isolate or D9THC distillate. As used herein, the term “D9THC isolate” means a form of D9THC being at least 99% pure D9THC and containing no other Cannabis plant compounds. As used herein, the term “D9THC distillate” means a form of D9THC being at least 80%-90% pure D9THC and containing one or more other Cannabis plant compounds. The compositions comprise about 1% to about 25%, preferably about 5% to about 20%, more preferably about 10% to about 15%, most preferably about 15% of the one or more cannabinoids, wherein the % by weight is based upon the total weight of the compositions each taken as 100% by weight.

In most embodiments, the compositions comprise at least one emulsifier. The at least one emulsifier may be any surfactant that is dispersible or soluble in the primary solvent, preferably a surfactant with a hydrophilic/lipophilic balance (HLB) value that is greater than about 6 and up to 20. As used herein, the term “HLB value” means the balance of the size and strength of the hydrophilic and lipophilic moieties of a surfactant molecule, and the HLB value scale ranges from 0 to 20. In other preferred embodiments, the at least one emulsifier may be an oil-in-water surfactant, preferably an oil-in-water surfactant selected from the group consisting of polyoxyethylene sorbitan derivatives, triethanolamine oleate, sodium oleate, and potassium oleate. Preferred polyoxyethylene sorbitan derivatives may include, but are not limited to, polyoxyethylene sorbitan monooleate (TWEEN 80), polyoxyethylene sorbitan monostearate (TWEEN 60), polyoxyethylene sorbitan monopalmitate (TWEEN 40), and polyoxyethylene sorbitan monolaurate (TWEEN 20). In most preferred embodiments, the at least one emulsifier comprises TWEEN 80. The compositions comprise about 1% to about 25%, preferably about 5% to about 20%, more preferably about 10% to about 15%, most preferably about 15% of the at least one emulsifier, wherein the % by weight is based upon the total weight of the compositions each taken as 100% by weight.

In most embodiments, the compositions comprise at least one co-solvent. The at least one co-solvent may be any solvent miscible or soluble with the primary solvent and with water, preferably a solvent that will not be substantially evaporated with the primary solvent in the drying process (i.e., not volatile). In preferred embodiments, the at least one co-solvent includes solvents selected from the group consisting of polyethylene glycol (PEG) and derivatives thereof, propylene glycol, glycerin, benzyl alcohol, benzaldehyde, dimethyl sulfoxide (DMSO), and N, N-dimethylacetamide (DMA). Preferred PEG derivatives may include, but are not limited to, derivatives in liquid form with lower volatility, such as PEG-400 or a PEG derivative with a molecular weight between about 100 and about 1000. The compositions comprise about 0.5% to about 5%, preferably about 2% to about 5%, more preferably about 3% to about 5%, most preferably about 3% of the at least one co-solvent, wherein the % by weight is based upon the total weight of the compositions each taken as 100% by weight.

In one or more embodiments, the compositions may further comprise at least one water-soluble bulking agent, which provides a solid granulate base for the composition. The at least one water-soluble bulking agent may be any material that is a water-soluble solid at ambient temperature, i.e., has a solubility greater than about 10% by weight in water at about 15° C. to about 25° C. In preferred embodiments, the at least one water-soluble bulking agent may be a polysaccharide material, preferably, comprised of one or more sugars. Preferred sugars include lactose, polyols (e.g., mannitol), polysaccharides (e.g., starch), and or water-soluble cellulosic materials (e.g., carboxymethylcellulose). It will be appreciated that these materials are also not soluble or have very limited solubility in the primary solvent, i.e., has a solubility less than about 2% by weight of the selected primary solvent at temperatures below about 60° C. The compositions comprise about 25% to about 90%, preferably about 30% to about 85%, more preferably about 45% to about 70%, most preferably about 67% of the at least one water-soluble bulking agent, wherein the % by weight is based upon the total weight of the compositions each taken as 100% by weight.

In preferred embodiments, the weight ratio of the one or more cannabinoids and the at least one emulsifier is about 2:1 to about 1:2, preferably about 1:1, the weight ratio of the one or more cannabinoids and the at least one solvent is about 1:3 to about 1:30, preferably about 1:5, and the weight ratio of the one or more cannabinoids and the at least one water-soluble bulking agent is about 3:1 to about 8:1, preferably about 4.5:1.

### 2. Primary Solvent

In one or more embodiments, the compositions comprise one or more cannabinoids, at least one emulsifier, and at least one co-solvent, which are each dissolved or dispersed in a primary solvent. The primary solvent comprises one or more organic solvents that function as a solvent to fully dissolve the one or more cannabinoids, the at least one emulsifier, and the at least one co-solvent, particularly the one or more cannabinoids. Preferably, the total amount of the primary solvent is in excess of the solubility of the one or more cannabinoids in the primary solvent. In preferred embodiments, the primary solvent is volatile so that it can be evaporated substantially from the composition at ambient temperature, i.e., about 15° C. to about 25° C., or at a temperature of up to about 90° C. to about 110° C., preferably about 100° C. If the composition is dried at a temperature above ambient temperature, a vacuum may also be used to increase the volatility of the primary solvent during the drying process. In the final composition, the primary solvents used are substantially evaporated to levels not analytically detectable or to measured values less than about 1% by weight of the composition. The primary solvent may be, but is not limited to, volatile alcohols, such as supercritical carbon dioxide, acetone, ethyl acetate, methylene chloride, methanol, ethanol, n-propanol, and/or isopropanol, preferably ethanol.

### 3. Other Ingredients

It will be appreciated that a number of other optional ingredients can be included in the compositions as well. Typical optional ingredients include those selected from the group consisting of water-insoluble cross-linked PVP (e.g., crospovidone), poloxamers, cyclodextrins, and phospholipids.

The compositions may be in the form of granules, compressed into tablets or soft chews, placed in capsules, powder sachets, mixed with dry tea or coffee blends, or incorporated with other powder mixes or pre-mixes intended for consumption. It will be appreciated that the compositions are suitable to administer to an animal. In some embodiments, the animal may be an animal species suitable for the household, such as canines, felines, birds, rabbits, ferrets, guinea pigs, small reptiles, and the like. In other embodiments, the animal may be poultry or livestock, such as horses, cattle, sheep, goats, pigs, and the like. In other embodiments, the animal may be a human being.

The compositions exhibit substantially greater water-soluble and water-dispersible properties in comparison to other Cannabis products, such as products made from the extract or isolate of Cannabis directly mixed with a surfactant. For example, it will be appreciated that, upon dispersing and/or solubilizing a quantity of the compositions in a liquid carrier, preferably water, at least 75% by weight of, preferably by weight of 85%, more preferably by weight of 95%, most preferably by weight of 99% of the one or more cannabinoids present in the composition are transferred into the liquid carrier and can be recovered therefrom. In addition, when an effective amount of the composition in a liquid carrier, preferably water, is consumed orally, at least about 50%, preferably at least about 65%, more preferably at least about 80% of the one or more cannabinoids within the composition is bioavailable within 1 hour after ingestion. As used herein, an “effective amount” refers to an amount capable of providing the desired bioavailable levels of the active compound (i.e., cannabinoid). In most preferred embodiments, when an effective amount is administered to an animal about 100 to about 140 ng/mL of the one or more cannabinoids are taken up into the animal's blood stream within 1 hour.

It will also be appreciated that the compositions are capable of forming a stable emulsion with a liquid carrier without heat, ultrasonication, spray drying, or high-pressure extrusion. The compositions allow lipophilic compounds, such as the one or more cannabinoids, to evenly disperse into the liquid carrier, without the formation of an unstable oil emulsion, insoluble cannabinoid particles (typically settling to the bottom of the liquid carrier), or an oily film (typically forming on the top of the liquid carrier).

The liquid carrier may be water, tea, coffee, milk, sports drinks, or alcoholic beverages, preferably water. When the composition is added to water, the composition can form a stable emulsion with water at a weight ratio of from about 1:25 to about 1:35, preferably 1:30. This stable emulsion can have a concentration of up to 4 mg/mL of the one or more cannabinoids in 4° C. water, in room temperature water, in 80° C. water with gentle stirring. It will be appreciated that this concentration of cannabinoids can be achieved without the use of additives, prolonged processing, or significant energy input, such as significant heat or high shear mixing.

### Methods of Making the Compositions

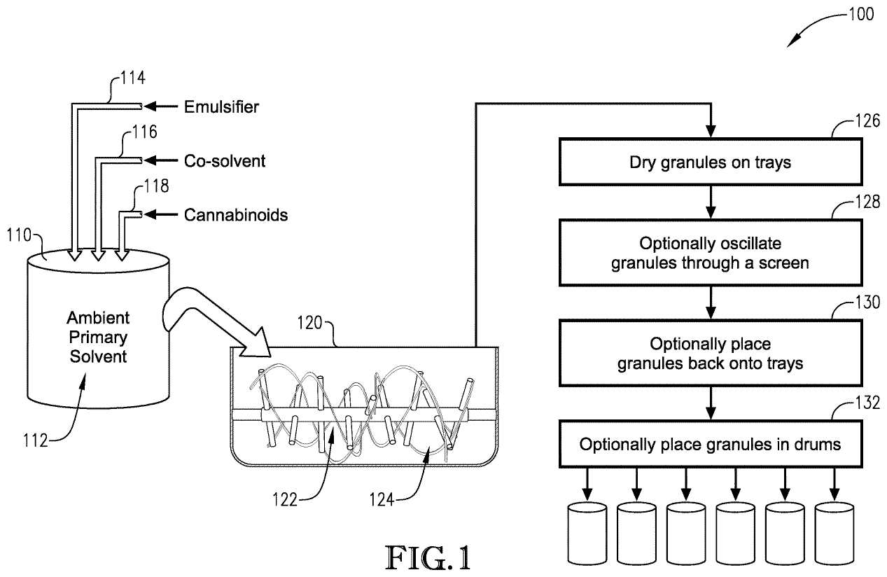

FIG. 1 is a process diagram illustrating a method of producing the compositions 100 according to one embodiment of the present invention. The compositions are generally produced by mixing a liquid phase 110 with a dry phase 120. The liquid phase 110 comprises the primary solvent 112, the at least one emulsifier 114, the at least one co-solvent 116, and the one or more cannabinoids 118. To form the liquid phase 110, the at least one emulsifier 114, the at least one co-solvent 116, and the one or more cannabinoids 118 in dry or liquid form, preferably dry form, are added to the primary solvent 112. In preferred embodiments, under ambient conditions, the liquid phase 110 is formed by first adding the least one emulsifier 114 and the co-solvent 116 to the primary solvent 112 and mixing until homogenous. Then, the one or more cannabinoids 118 are added to this mixture and stirred until homogenous. In most preferred embodiments, under ambient conditions, the at least one emulsifier 114 is first added and mixed into the primary solvent 112, followed by the at least one co-solvent 116, which is added and mixed into the primary solvent/emulsifier mixture. Then, the one or more cannabinoids 118 are added to the primary solvent/emulsifier/co-solvent mixture and stirred until homogenous. As used herein, the phrase “ambient conditions” refers to the general environmental conditions of the area in which the method is being performed, with no external heat or cooling provided.

The liquid phase 110 is then added to the dry phase 120 and mixed until uniform to form the composition. As used herein, the term “dry phase” 120 means a solid, water-soluble material 122, such as a bulking agent, to which the liquid phase 110 is added. The composition comprises from about 30% to about 50% by weight of the liquid phase 110 and from about 50% to about 70% by weight of the dry phase 120, wherein each % by weight is based upon the total weight of the composition formed. In preferred embodiments, the weight ratio of the liquid phase 110 and the dry phase 120 is about 1:1.5 to about 1:2.5, preferably about 1:1.6. The liquid phase 110 comprises about 15% to about 30% by weight of at least one primary solvent 112, about 25% to about 45% by weight of at least one emulsifier 114, about 5% to about 10% by weight of at least one co-solvent 116, and about 25% to about 45% by weight of one or more cannabinoids 118, wherein each % by weight is based upon the total weight of the liquid phase 110. To ensure a uniform mixture, in preferred embodiments, the dry phase 120 and liquid phase 110 can be mixed under low shear using a suitable mixer 124, such as a ribbon blender.

Once the composition is formed, the composition is dried on trays 126 under ambient conditions until the primary solvent 112 is substantially evaporated, preferably until less than 0.5% by weight of primary solvent 112 remains in the composition. In other embodiments, the composition may be further dried at a temperature of up to about 90° C. to about 110° C., preferably 100° C. using methods known in the art, such as vacuum dryers, hot air dryers, and the like. In preferred embodiments, the compositions are not mixed (i.e., aggressive mixing or shear) after drying to avoid transitioning to a semi-solid state, which has reduced water recovery of cannabinoids than that of the dried composition.

To break up agglomerates, in most preferred embodiments, the compositions may be oscillated through a mesh screen 128, preferably an 8-mesh screen. In some embodiments, if the composition needs to be dried further, the composition may be optionally dried again on trays 130 after oscillating. Finally, if desired, the granules can be stored in drums 132.

### Liquid Suspensions Containing the Compositions

Once the compositions are produced according to the methods described above, the compositions may be added to a liquid carrier to form a liquid suspension. In some embodiments, the liquid suspension comprises about 1 part by weight of the composition dispersed in about 20 parts to about 80 parts, preferably about 40 parts to about 60 parts, more preferably about 50 parts by weight of the liquid carrier. At these amounts, the liquid suspensions can have a concentration of about 0.1 mg/mL to about 5 mg/mL, preferably about 2 mg/mL to about 4 mg/mL of cannabinoids. The concentration of cannabinoids in the liquid suspensions is not negatively impacted by further filtration through a coffee filter, stainless steel filter, or the like.

The liquid carrier may be water, tea, coffee, milk, sports drinks, or alcoholic beverages, preferably water. It will be appreciated that the liquid suspensions are substantially free of oil.

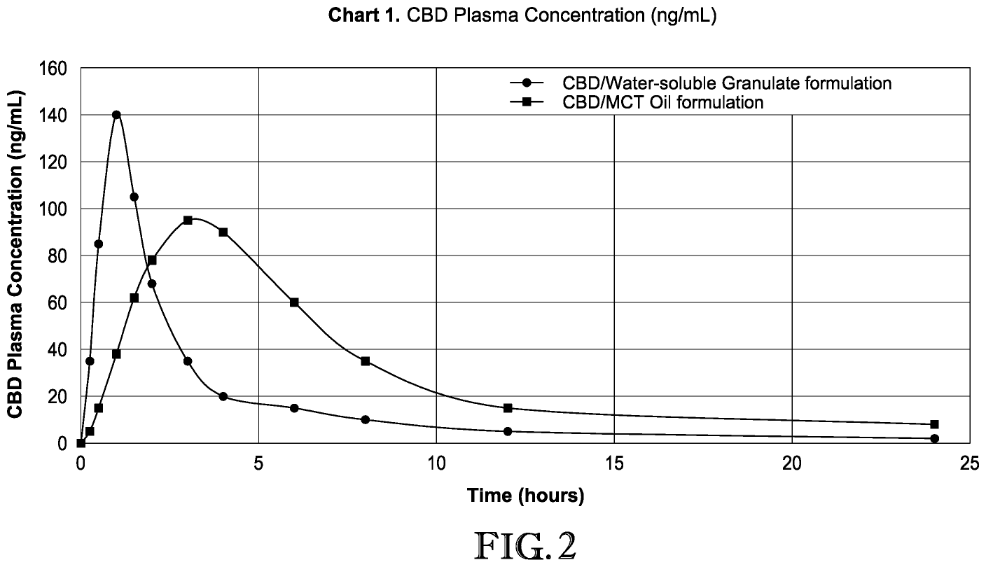

It will also be appreciated that, when an effective amount of the composition in a liquid carrier, preferably water, is administered, at least about 50%, preferably at least about 65%, more preferably at least about 80% of the one or more cannabinoids within the composition is bioavailable within 1 hour after ingestion. In most preferred embodiments, when an effective amount is administered to an animal, preferably a canine, about 140 ng/mL of the one or more cannabinoids are taken up into the animal's blood stream within 1 hour. For example, as shown in FIG. 2, the liquid suspensions comprising the compositions exhibit a faster in vivo absorption than the similarly dosed isolate administered dissolved in a compounded medium chain triglycerides (MCT) oil base.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### CBD Formulation Preparation

A cannabidiol (CBD) formulation was prepared using the ingredients and quantities shown in Table 1. FIG. 1 provides an illustration of the preparation method. First, TWEEN 80 was added to ethanol and stirred until dissolved. Then, PEG-400 was added to the ethanol and TWEEN 80 mixture and stirred until dissolved, forming a liquid phase. Next, the CBD isolate was added as a powder to the liquid phase and mixed until visually dissolved. After dissolving the CBD isolate, the liquid phase was transferred to a ribbon blender and mixed thoroughly with mannitol under low shear at about 60 to about 100 rpm to ensure uniform mixing. Under ambient conditions, the CBD formulation was air dried on trays to remove the ethanol. Gas chromatography confirmed that the dried CBD formulation contained less than 0.5% by weight of ethanol, as shown in Table 2. Finally, the dried CBD formulation was oscillated through an 8-mesh screen to break-up agglomerates, after which the formulation was packaged and sealed in lined drums.

TABLE 1

| | Quantity |
|---|---|
| Ingredient | (parts by weight) |
| Alcohol, USP (Ethyl Alcohol) | 10 |
| TWEEN 80 | 15 |
| PEG-400 | 3 |
| CBD Isolate (99% purity) | 15 |
| Mannitol (dry powder base) | 67 |

TABLE 2
Dried CBD Formulation

| Ingredient | Quantity* |
|---|---|
| Alcohol, USP (Ethyl Alcohol) | 0% |
| TWEEN 80 | 15.0% |
| PEG-400 | 3.0% |
| CBD Isolate (99% purity) | 15.0% |
| Mannitol (dry powder base) | 67.0% |

*% by weight based on the combined weight of all ingredients taken as 100% by weight.

### Example 2

### Water Recovery and Potency Study

A laboratory scale batch of a CBD formulation was prepared using the same ingredients and preparation method described in Example 1. 500 mg of the formulation was mixed into 100 mL of water. A high-performance liquid chromatography (HPLC) analysis was performed to identify the water recovery of the CBD in the water, and a CBD assay was performed to identify the potency of the CBD in the water. The results from the HPLC analysis and CBD assay are shown in Table 3.

TABLE 3
HPLC Analysis and CBD Assay Results

| Test | Result |
|---|---|
| HPLC Analysis | 96.4% recovery in water |
| CBD Assay | 0.1498 mg/mg (15% w/w) |

The results revealed that the formulation described in Example 1 has a high water recovery of CBD and a high potency level when mixed with water. Notably, these results were achieved without the use of additives, prolonged processing, or significant energy input, such as heat or high shear mixing.

### Example 3

A study was conducted to determine whether water containing the CBD formulation is suitable for further filtration and contains recoverable CBD after filtration.

### Coffee Filtration Study

A CBD formulation was prepared using the same ingredients and preparation method described in Example 1. A tabletop Keurig® coffee maker was used to heat and dispense ˜230 mL of water through a re-usable stainless steel (80-mesh) coffee filter and a household paper coffee filter (pore size=˜20 um). ˜550 mg of the formulation (i.e., 82.5 mg of CBD isolate) was placed into each filter, and the heated water was poured through using the tabletop coffee maker. The temperature of the water was measured to be ˜75° C. The CBD filtered water was cooled to room temperature for ˜15-20 minutes and was stirred for 10 minutes. After stirring for 10 minutes, a sample was collected for HPLC analysis.

The HPLC results revealed that the water recovery of CBD in the filtered water was ˜90%, indicating that the concentration of CBD in the water was 0.323 mg/ml. Notably, the test identified that CBD was able to pass easily through the re-usable stainless steel mesh coffee filter as well as the household paper coffee filter with less than a 5% loss of cannabinoids as compared to non-filtered CBD water (˜0.346 mg/ml CBD).

### Example 4

### Preparation Method and its Effect on Water Recovery

A study was conducted to illustrate the effect of the preparation method on the final performance of the formulation in water.

CBD Combined with Dry Phase Before Liquid Phase

15 g of CBD was added as a dry blend to 15 g of a dry phase, Prosolv (a combination of microcrystalline cellulose and colloidal silicon dioxide). In a separate container, various amounts of co-solvent PEG-200DL were added to ethanol as shown in Table 4. These solutions were sprayed onto each of the blends of CBD/Prosolv while mixing to form a granulation. Under ambient conditions, the formulation was dried on trays overnight.

Once the formulation was dry, an additional amount of PEG200DL was added with additional ethanol. Next, 500 mg of the formulation was added to 30 mL of water, and HPLC analysis was used to test the water recovery of CBD in the suspensions. The results revealed that the water recovery of the CBD in water was ˜30%.

TABLE 4

| Suspen- | Ethanol | PEG200DL | Sample Size | Additional | Additional |
|---|---|---|---|---|---|
| sions | (g) | (g) | (g) | Ethanol | PEG200DL |
| A | 6.5 | N/A | N/A | N/A | N/A |
| B | 4.11 | 1.5 | 5 | 1.37 | 0.5 |
| C | 4.16 | 0.78 | N/A | N/A | N/A |

These results reveal that, if CBD is mixed with the dry phase without first being dissolved in the liquid phase, the resulting formulation will be less soluble in water.

### CBD Added to Emulsifier or Co-Solvent and Water

A study was conducted with several different emulsifiers or co-solvents added to water and CBD powder.

### Experiment 1

In a first experiment, five 50 mL centrifuge tubes were each filled with 40 mL of water and 200 mg of CBD powder. Then, increasing amounts of PEG-400 (65 mg, 130 mg, 195 mg, 260 mg, and 650 mg) were added respectively to each centrifuge tube, which were then placed on a tabletop shaker and mechanically shaken on high for five minutes. All tubes were observed to have some CBD powder at the bottom, with the tube containing 650 mg of PEG-400 having the least amount of residue. No emulsion, however, was immediately formed after mixing.

### Experiment 2

In a second experiment, four 50 mL centrifuge tubes were each filled with 40 mL of water and 200 mg of CBD powder. Then, increasing amounts of TWEEN 80 (160 mg, 320 mg, 800 mg, and 1600 mg) were added respectively to each centrifuge tube, which were then placed on a tabletop shaker and mechanically shaken on high for five minutes. TWEEN 80 formed a big clump when mixed with the water and CBD, and as a result, no emulsion was formed in any tube. Further, CBD powder was still floating around in each tube.

### Experiment 3

In a third experiment, four 50 mL centrifuge tubes were each filled with 40 mL of water and 200 mg of CBD powder. Then, increasing amounts of sodium dodecyl sulfate (SDS) (10 mg, 25 mg, 50 mg, and 100 mg) were added respectively to each centrifuge tube, which were then placed on a tabletop shaker and mechanically shaken on high for five minutes. SDS produced a foam when mixed with the water and CBD powder, but no emulsion was formed in any tube. Further, CBD residue was observed on the bottom of each tube.

### Experiment 4

Finally, in a fourth experiment, four 50 mL centrifuge tubes were each filled with 40 mL of water and 200 mg of CBD powder. Then, increasing amounts of glycerol monostearate (GMS) (10 mg, 25 mg, 50 mg, and 100 mg) were added respectively to each centrifuge tube, which were then placed on a tabletop shaker and mechanically shaken on high for five minutes. GMS did not mix with the water and CBD powder, and as a result, no emulsion was formed in any tube. Further, CBD residue was observed on the bottom and sides of each tube.

In view of the foregoing experiments, the results from the study revealed that PEG-400, TWEEN 80, SDS, and GMS were not able to create a stable emulsion with just CBD powder and water.

### Example 5

### Emulsifier and its Effect on Water Recovery

### Effect of the Ratio of CBD to Emulsifier

A study was conducted to establish the best ratio of emulsifier to CBD, and different weight to weight ratios of CBD to emulsifier were evaluated to determine the best recovery in water.

### Experiment 1

In a first experiment, a formulation was prepared using the ingredients and amounts shown in Table 5. First, TWEEN 80 was added to ethanol and stirred until dissolved. Then, PEG-400 was added to the ethanol and TWEEN 80 mixture and stirred until dissolved, forming a liquid phase. Next, the CBD isolate as added as a powder to the liquid phase and mixed until visually dissolved. After dissolving the CBD isolate, the liquid phase was mixed thoroughly with lactose until granules began to form. Under ambient conditions, the CBD formulation was then air dried overnight on trays to remove the ethanol. The dried CBD formulation was sifted through a screen to break-up agglomerates. The dried CBD formulation formed well and quickly.

To test the water recovery of the CBD in the formulation, 500 mg of the formulation was added to 100 mL of water. The formulation dispersed in the water and formed cloudy solution. After several minutes, the formulation settled at the bottom, but was in the form of fine particles, not clumps.

TABLE 5
1:1 CBD/TWEEN 80 Ratio

| Ingredient | Quantity (g) |
|---|---|
| Alcohol, USP (Ethyl Alcohol) | 10.0 |
| TWEEN 80 | 5.0 |
| PEG-400 | 1.0 |
| CBD Isolate (99% purity) | 5.0 |
| Lactose (dry powder base) | 89.0 |

### Experiment 2

In a second experiment, a formulation was prepared using the ingredients and amounts shown in Table 6 and using the preparation method described above in Experiment 1. The dried CBD formulation formed well and quickly.

To test the water recovery of the CBD in the formulation, 500 mg of the formulation was added to 100 mL of water. The formulation dispersed in the water but began to clump at stirring. Some floating also occurred.

TABLE 6
5:1 CBD/TWEEN 80 Ratio

| Ingredient | Quantity (g) |
|---|---|
| Alcohol, USP (Ethyl Alcohol) | 10.0 |
| TWEEN 80 | 1.0 |
| PEG-400 | 1.0 |
| CBD Isolate (99% purity) | 5.0 |
| Lactose (dry powder) | 93.0 |

### Experiment 3

In a third experiment, a formulation was prepared using the ingredients and amounts shown in Table 7 and using the preparation method described above in Experiment 1. The dried CBD formulation formed well and quickly.

To test the water recovery of the CBD in the formulation, 500 mg of the formulation was added to 100 mL of water. The formulation dispersed in the water but began to clump at stirring. Some floating also occurred.

TABLE 7
10:1 CBD/TWEEN 80 Ratio with 1 g of PEG 400

| Ingredient | Quantity (g) |
|---|---|
| Alcohol, USP (Ethyl Alcohol) | 13.0 |
| TWEEN 80 | 1.5 |
| PEG-400 | 1.0 |
| CBD Isolate (99% purity) | 10.0 |
| Lactose (dry powder) | 87.5 |

### Experiment 4

Finally, in a fourth experiment, a formulation was prepared using the ingredients and amounts shown in Table 8 and using the preparation method described above in Experiment 1. The dried CBD formulation formed well and quickly.

To test the water recovery of the CBD in the formulation, 500 mg of the formulation was added to 100 mL of water. The formulation dispersed in the water but began to clump at stirring. Most of the formulation sunk to the bottom.

TABLE 8
10:1 CBD/TWEEN 80 Ratio with 2 g of PEG 400

| Ingredient | Quantity (g) |
|---|---|
| Alcohol, USP (Ethyl Alcohol) | 13.0 |
| TWEEN 80 | 1.5 |
| PEG-400 | 2.0 |
| CBD Isolate (99% purity) | 10.0 |
| Lactose (dry powder) | 86.5 |

The results of this study reveal that the 1:1 CBD/TWEEN 80 ratio had the most positive effect on the water recovery of the CBD in water.

### Effect of Emulsifier or Co-Solvent Selection

A study was conducted to identify the best emulsifier or co-solvent for the formulation, and different weight to weight ratios of CBD to emulsifier were evaluated to determine the best recovery in water.

### Experiment 1

In a first experiment, a formulation was prepared using the ingredients and amounts shown in Table 9. In this formulation, the amount of emulsifiers were reduced to 20% w/w with CBD. To prepare the formulation, ethanol, GMS (an emulsifier with a low HLB value), TWEEN 80 (an emulsifier with a high HLB value), and PEG-400 were combined and mixed until homogenous, forming a liquid phase. Next, the CBD isolate was added as a powder to the liquid phase and mixed until visually dissolved. After dissolving the CBD isolate, the liquid phase was mixed thoroughly with Mannogem XL (spray-dried mannitol) and Crospovidone, which is a super disintegrant used in rapidly dissolving tablets. Under ambient conditions, the CBD formulation was air dried on trays to remove the ethanol.

Before the drying step, the CBD formulation had a sticky texture, but was the least sticky of the formulations made in Experiments 1-3. Once the formulation was dried overnight, 500 mg of the formulation was added to 100 mL of water, and HPLC analysis was used to test the water recovery of the CBD in the formulation. The results revealed that the water recovery of the CBD in water was ˜45%.

TABLE 9

| | Quantity | Wt (g) for a 150 g |
|---|---|---|
| Ingredient | (parts by weight) | dried formulation |
| Glycerol Monostearate (GMS) | 3.9 | 5.9 |
| Alcohol, USP (Ethyl Alcohol) | 13.0 | 19.5 |
| TWEEN 80 | 1.8 | 2.7 |
| CBD Isolate (99% purity) | 22.2 | 33.3 |
| PEG-400 | 3.2 | 4.8 |
| Mannogem XL (SPI Pharma) | 63.0 | 94.5 |
| Crospovidone (water-insoluble | 5.9 | 8.9 |
| cross-linked PVP) | | |

### Experiment 2

In a second experiment, a formulation was prepared using the ingredients and amounts shown in Table 10 and the preparation method described in Experiment 1. In this formulation, the amount of emulsifiers were reduced to 15% w/w with CBD.

Like the formulation in Experiment 1, the CBD formulation had a sticky texture before drying. Once the formulation was dried overnight, 500 mg of the formulation was added to 100 mL of water, and HPLC analysis was used to test the water recovery of the CBD in the formulation. The results revealed that the water recovery of the CBD in water was ˜45%.

TABLE 10

| | Quantity | Wt (g) for a 150 g |
|---|---|---|
| Ingredient | (parts by weight) | dried formulation |
| Glycerol Monostearate (GMS) | 4.2 | 6.2 |
| Alcohol, USP (Ethyl Alcohol) | 13.0 | 19.5 |
| TWEEN 80 | 1.9 | 2.8 |
| CBD Isolate (99% purity) | 22.2 | 33.3 |
| PEG-400 | 3.4 | 5.1 |
| Mannogem XL (SPI Pharma) | 62.4 | 93.6 |
| Crospovidone (water-insoluble | 5.9 | 8.9 |
| cross-linked PVP) | | |

### Experiment 3

Finally, in a third experiment, a formulation was prepared using the ingredients and amounts shown in Table 11 and the preparation method described in Experiment 1. In this formulation, the amount of emulsifiers were reduced to 10% w/w with CBD.

Notably, even after the drying step, the formulation was slightly sticky. Despite this, 500 mg of the formulation was added to 100 mL of water, and HPLC analysis was used to test the water recovery of the CBD in the formulation. The results revealed that the water recovery of the CBD in water was ˜45%.

TABLE 11

| | Quantity | Wt (g) for a 150 g |
|---|---|---|
| Ingredient | (parts by weight) | dried formulation |
| Glycerol Monostearate (GMS) | 4.4 | 6.6 |
| Alcohol, USP (Ethyl Alcohol) | 13.0 | 19.5 |
| TWEEN 80 | 2.0 | 3.0 |
| CBD Isolate (99% purity) | 22.2 | 33.3 |
| PEG-400 | 3.6 | 5.4 |
| Mannogem XL (SPI Pharma) | 61.9 | 92.9 |
| Crospovidone (water-insoluble | 5.9 | 8.9 |
| cross-linked PVP) | | |

This study illustrates that, when selecting an emulsifier, the following should be taken consideration: (1) its solubility in the solvent, (2) its physical state at room temperature (solid vs liquid), and (3) its influence of the physical state of the granulate once the solvent is dried off. Some emulsifiers, like GMS, require higher heat to dissolve in the solvent phase because they are solid at room temperature (unless dissolved in high amounts of oils), and this could cause precipitation during manufacturing, resulting in a less desirable water recovery and altering the overall physical appearance of the granulation. For example, as illustrated above, instead of a free-flowing powder, a hard granule or clump is formed.

### Example 6

### Bioavailability of the Formulation

An 18-dog crossover pharmacokinetic study with beagle dogs was used to evaluate the bioavailability of the CBD granulate formulation (prepared using the same ingredients and preparation method as described in Example 1) in a water vehicle against a separate preparation of the same CBD isolate dissolved and dosed in a medium chain triglycerides (MCT) oil vehicle. Each approximately 10 kg dog was accurately weighed the morning of dosing, and the volume of each formulation was adjusted to deliver a dose of 2 mg/kg.

All statistical analyses were studied with the individual animal as the experimental unit. Using the blood sample assay data, the area under the plasma-concentration-vs.-time curve (AUC_{0-LLOQ} and AUC_{0-∞}), maximum observed drug concentration (Cₘₐₓ), and time corresponding to Cₘₐₓ (Tₘₐₓ) were calculated for each animal in each period. AUC was evaluated as a measure of bioavailability, Cₘₐₓ and Tₘₐₓ as measures of the absorption rate, and the adequacy of the sampling profile was verified if AUC_{0-LLOQ}/AUC_{0-∞} was at least 0.80. Nominal sampling times were used in the AUC estimation regardless of variation (if any) from the authorized sampling time allowance. Log (ln) transformed AUC_{0-LOQ} and log Cₘₐₓ were statistically evaluated via analysis of variance (ANOVA) appropriate for a single-dose, three-treatment, three-period crossover design using the MIXED procedure in the Statistical Analysis System (SAS) (SAS Institute, Cary, N.C., version 9.4). Formulation, sequence, and period were included in the model as fixed effects.

The main effect of formulation was significant for both AUC_{0-LOQ} and Cₘₐₓ. As illustrated in Table 12, AUC_{0-LOQ} values in CBD granulate formulation were lower than values in CBD/MCT oil formulation (P<0.05), and Cₘₐₓ values in CBD/MCT oil formulation were significantly lower than values in CBD granulate formulation (P<0.05). Furthermore, the observed time (Tₘₐₓ) to Cₘₐₓ in CBD granulate formulation was much earlier than in CBD/MCT oil formulation.

TABLE 12
Formula comparison of each of the primary
outcome pharmacokinetic parameters

| | | LS means (Geometric means) | |
|---|---|---|---|
| | | CBD/Water-soluble | CBD/MCT oil |
| Parameter | Units | granulate formulation | formulation |
| AUC_{0-LOQ} | h*ng/mL | 256* | 469* |
| Cₘₐₓ | ng/mL | 113* | 74* |
| Tₘₐₓ | hours post-dose | 1.0 | 3.67 |

*Values significantly different at P < 0.05

This study demonstrated, with statistical significance, that the CBD granulate formulation provides a much faster absorption of the CBD compound and a peak blood level concentration at 52.7% higher than the maximum concentration observed in the CBD/MCT oil formulation. FIG. 2 illustrates the average plasma concentrations across all dogs for each formulation.

We claim:

1. A composition comprising:
about 5% to about 20% by weight of one or more cannabinoids;
about 5% to about 20% by weight of at least one emulsifier;
about 0.5% to about 5% by weight of at least one solvent; and
about 30% to about 85% by weight of at least one water-soluble bulking agent,
wherein each % by weight is based upon the total weight of said composition taken as 100% by weight.

2. The composition of claim 1, wherein the one or more cannabinoids are exogenous cannabinoids or endogenous cannabinoids.

3. The composition of claim 2, wherein the exogenous cannabinoids are natural exogenous cannabinoids extracted or isolated from the Cannabis plant.

4. The composition of claim 2, wherein the endogenous cannabinoids are selected from the group consisting of arachidonoylethanolamide (anandamide), 2-arachidonoyl glycerol (2-AG), palmitoylethanolamide, and methanandamide.

5. The composition of claim 1, wherein in the one or more cannabinoids is in dry form or liquid form.

6. The composition of claim 1, wherein the one or more cannabinoids is selected from the group consisting of cannabidiols (CBD), cannabigerols, cannabichromenes, cannabinols, delta 8-tetrahydrocannabinols, delta 9-tetrahydrocannabinols and delta 10-tetrahydrocannabinols.

7. The composition of claim 1, wherein the one or more cannabinoids is CBD, wherein the form of CBD is CBD isolate, broad-spectrum CBD, or full-spectrum CBD.

8. The composition of claim 1, wherein the at least one emulsifier is a surfactant having an HLB value that is in the range of about 6 to about 20.

10. The composition of claim 1, wherein the composition is in the form of granules, a powder, tablets, or soft chews.

11. The composition of claim 1, wherein the composition is capable of forming a stable emulsion with water at a weight ratio of from about 1:25 to about 1:35.

12. The composition of claim 1, wherein the weight ratio of the one or more cannabinoids and the at least one emulsifier is about 2:1 to about 1:2.

13. The composition of claim 1, wherein at least about 50% of the one or more cannabinoids within the composition is bioavailable within 1 hour after ingestion.

14. The composition of claim 1, when fed an effective amount of the composition to a canine, about 100 to about 140 ng/mL of the one or more cannabinoids are present in the canine's blood within 1 hour.

15. The composition of claim 1, wherein upon dispersing and/or solubilizing a quantity of the composition in water at least 75% by weight of the one or more cannabinoids present in the composition are transferred into the water and can be recovered therefrom.

16. A method of producing a composition comprising:
(a) combining at least one primary solvent, at least one emulsifier, at least one co-solvent, and one or more cannabinoids together to form a liquid phase;
(b) mixing the liquid phase with a dry phase to form a cannabinoid composition, the dry phase comprising at least one water-soluble bulking agent; and
(c) drying the composition.

17. A method according to claim 16, wherein step (a) further comprises:
mixing the at least one primary solvent with the at least one emulsifier and the at least one co-solvent to form a portion of the liquid phase; and
mixing the one or more cannabinoids to the portion of the liquid phase, thereby forming the liquid phase.

18. A method according to claim 16, wherein the cannabinoid composition formed in step (b) comprises from about 30% to about 50% by weight of the liquid phase and from about 50% to about 70% by weight of the dry phase, wherein each % by weight is based upon the total weight of the cannabinoid composition formed in step (b).

19. A method according to claim 16, wherein the liquid phase comprises:
about 15% to about 30% by weight of at least one primary solvent,
about 25% to about 45% by weight of at least one emulsifier,
about 5% to about 10% by weight of at least one co-solvent, and
about 25% to about 45% by weight of one or more cannabinoids;
wherein each % by weight is based upon the total weight of the liquid phase.

20. A method according to claim 16, wherein the method further comprises:
(d) after step (c), oscillating the composition through a mesh screen to form a granular composition.

21. A liquid suspension comprising about 1 part by weight of the composition of claim 1 dispersed in no more than about 50 parts by weight of a liquid carrier, wherein the suspension has a concentration of about 0.1 mg/mL to about 5 mg/mL of cannabinoids.

22. The liquid suspension of claim 21, wherein the liquid carrier is water.

23. The liquid suspension of claim 21, wherein the suspension is substantially free of oil.

24. A method of administering cannabinoids to an animal, the method comprising feeding the animal an effective amount of the composition of claim 1 or the liquid suspension of claim 21.

25. A method according to claim 24, wherein the animal is a canine or a feline.
